(19) United States
(12) Patent Application Publication
(10) Pub. No.: US 2005/0277561 A1
(43) Pub. Date: Dec. 15, 2005

(54) Soap composition

(75) Inventors: Joseph-Jeanne-Marie Hendrickx, Steenokkerzeel (BE); Johannes Helmond, Gouda (NL)

Correspondence Address: MAYER, BROWN, ROWE & MAW LLP, 1909 K STREET, N.W., WASHINGTON, DC, 20006, US

(73) Assignees: Imperial Chemical Industries PLC, London (GB); Unichema Chemie B.V., Gouda (NL)

(21) Appl. No.: 11/010,441

(22) Filed: Dec. 14, 2004

Related U.S. Application Data
(63) Continuation of application No. 10/481,753, now abandoned.

(30) Foreign Application Priority Data
Jun. 29, 2001 (GB) 0115942.5
Jun. 17, 2002 (WO) GB02/002712

(51) Int. Cl. A61K 7/50

(52) U.S. Cl. 510/141

(57) ABSTRACT

A soap composition containing: (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water. The soap composition is particularly suitable for use in forming a translucent or transparent soap bar.

### FIELD OF INVENTION

The present invention relates to a soap composition, and in particular to a transparent or translucent soap composition.

### BACKGROUND

A wide range of soap compositions, used to make soap bars or tablets, particularly toilet soap tablets for personal washing, are known in the art. There is a continual need to improve the properties of the soap composition or soap tablet, such as ability to lather, hardness, reduction in mush (softening when left standing in water), feel and moisturisation. It can be difficult to obtain a soap composition having all or most of the aforementioned properties. Soap compositions can be either opaque, translucent or transparent, and it can be particularly difficult to achieve the aforementioned properties whilst maintaining or improving the translucency or transparency of a soap composition.

### PRIOR ART

GB-1417183 and GB-1487552 both disclose detergent bars containing a water soluble lactate salt, which together with a water soluble glutamate salt, act as moisturising components.

U.S. Pat. No. 4,297,230 is directed to a transparent soap bar containing potassium soap and chloride anions.

WO 99/42554 is directed to a soap bar containing 30-60% by weight of alkali metal salt of a defined fatty acid mixture, 3-35% by weight of fatty acid, 2-25% by weight of structurant and the remainder water.

U.S. Pat. No. 6,218,348 claims a process of making a soap bar which contains polyalkylene glycol having a molecular weight in the range from 400 to 25,000.

### SUMMARY OF THE INVENTION

We have now surprisingly discovered a soap composition which overcomes or significantly reduces at least one of the aforementioned problems.

Accordingly, the present invention provides a soap composition comprising (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water.

The invention also provides a soap tablet or bar comprising (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water.

The invention further provides the use of a soap composition comprising (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water, to form a transparent and/or translucent soap tablet or bar.

The soap composition according to the present invention may be opaque, translucent or transparent, and is preferably transparent or translucent. By “opaque” is meant having the property of preventing the transmission of light so that objects placed behind an opaque soap cannot be seen. By “transparent” is meant having the property of transmitting light without appreciable scattering, so that objects placed behind a transparent soap are entirely visible and can easily be discerned. By “translucent” is meant having the property of allowing light to pass through partially or diffusely so that objects placed behind a translucent soap tablet cannot clearly be distinguished (therefore also called partly transparent or semi-transparent). The amount of light transmitted is, of course, dependent upon the thickness of the soap, and in the present context soap of 20 mm thickness was used as standard.

The soap composition preferably comprises in the range from 55 to 85%, more preferably 60 to 80%, particularly 63 to 75%, and especially 65 to 70% by weight of alkali metal soap of C₈-C₂₄ fatty acids, based on the total weight of the composition.

Fatty acids, suitable for use herein, can be obtained from natural sources such as, for instance, plant or animal esters (eg palm oil, palm kernel oil, coconut oil, babassu oil, soybean oil, castor oil, tallow, whale or fish oils, grease, lard, and mixtures thereof). The fatty acids can also be synthetically prepared (eg, by the oxidation of petroleum, or by the hydrogenation of carbon monoxide by the Fischer-Tropsch process). Resin acids, such as those present in tall oil, may be used. Naphthenic acids are also suitable.

Alkali metal soaps, such as sodium and potassium soaps, can be made by direct saponification of the fats and oils or by the neutralization of the free fatty adds which are prepared in a separate manufacturing process. Particularly preferred in the present invention are the sodium soaps, but small amounts, suitably less than 10%, preferably less than 8%, more preferably less than 5%, and particularly less than 1% by weight of non-sodium soaps, such as potassium soaps, magnesium soaps, ammonium soaps and/or alkanolamine soaps, and especially potassium soaps, may also be present. In a particularly preferred embodiment of the invention, the soap composition comprises substantially no potassium soaps. Alkali metal salts of mixtures of fatty acids derived from palm oil and palm kernel oil, eg sodium palm oil soaps and sodium palm kernel oil soaps, are preferred.

The term “palm oil” is used herein in to mean fatty acid mixtures having an approximate by weight carbon chain length distribution of 2.5% C₁₄, 29% C₁₆, 23%. C₁₈, 2% palmitoleic, 41.5% oleic and 3% linoleic acids (the first three fatty acids listed being saturated). Other sources having similar carbon chain length distributions, such as fatty acids derived from various tallows and lard, may also be used instead of or in addition to palm oil fatty acids.

The term “palm kernel oil” is used herein to mean fatty acid mixtures having an approximate by weight carbon chain length distribution of: 8% C₈, 7% C₁₀, 48% C₁₂, 17% C₁₄, 8% C₁₆, 2% C₁₈, 7% oleic and 2% linoleic acids (the first six fatty acids listed being saturated). Other sources having similar carbon chain length distributions, such as coconut oil and babassu kernel oil, may also be used instead of or in addition to palm kernel oil fatty acids.

The soap component preferably comprises in the range from (i) 10 to 98%, more preferably 40 to 95%, particularly 60 to 90%, and especially 75 to 85% by weight of soaps having in the range from 16 to 20 carbon atoms (preferably derived from palm oil fatty acids), and (ii) 2 to 90%, more preferably 5 to 60%, particularly 10 to 40%, and especially 15 to 25% by weight of soaps having in the range from 8 to 14 carbon atoms (preferably derived from palm kernel oil fatty acids and/or coconut oil).

The soap composition may also comprise a minor amount of one or more synthetic or non-soap detergents, which may be of the anionic, nonionic, amphoteric or cationic type, or mixtures thereof. Preferably less than 25%, more preferably less than 15%, particularly less than 10%, and especially less than 5% by weight, based on the total weight of the composition is non-soap detergent. In a particularly preferred embodiment of the invention, the soap composition comprises substantially no non-soap detergent.

Suitable non-soap detergents include (i) anionic detergents such as the alkyl aryl sulphonates, such as C₁₀-C₂₂ alkyl benzene sulphonates; the olefin sulphonate salts; the C₁₀-C₂₀ paraffin sulphonate salts; the C₈-C₂₂ fatty acyl sarcosinates; the C₈-C₂₂ fatty acyl isethionates and C₈-C₂₂ fatty acyl N-methyl taurides; and C₈-C₂₂ fatty acid alkanol amides; the C₈-C₂₀ alkyl sulphates and the sulphate esters of the reaction product of 1-20 moles of alkylene oxide with 2 to 5 carbon atoms and a saturated straight- or branched-chain aliphatic monohydric C₈-C₂₀ alcohol, such as sodium lauryl ether sulphate, (ii) nonionic detergents such as the reaction products of 1-50 mole of C₂-C₄ alkylene oxide with C₈-C₂₀ primary or secondary alkanols, with dihydric alcohols, and the like, (iii) amphoteric detergents such as the alkyl-β-iminodipropionates, and long-chain imidazole derivatives, such as imidazolinium betaines, and (iv) cationic detergents such as quaternary ammonium compounds, such as stearyl dimethyl benzyl ammonium chloride, and the like.

The concentration of polyols or polyhydric alcohols in the soap composition according to the present invention is preferably in the range from 5 to 20%, more preferably 8 to 18%, particularly 10 to 16%, and especially 12 to 14% by weight based on the total weight of the composition.

The molecular weight of the polyol is preferably less than 300, more preferably in the range from 50 to 270, particularly 80 to 220, and especially 90 to 200. Suitable polyols include sugar alcohols such as sorbitol, mannitol; (poly)glycols such as (poly)ethylene glycol, (poly)propylene glycol; and other C₃-C₆ polyols containing from 3 to 6 hydroxyl groups such as trimethylolpropane, trimethylolethane, and glycerine. Sugar alcohols, particularly sorbitol, are preferred. Mixtures of any two or more of the aforementioned materials may also be employed.

In a preferred embodiment, the soap composition according to the present invention preferably comprises a polyol mixture of a sugar alcohol, particularly sorbitol, and glycerine. The concentration of sugar alcohol in the soap composition according to the present invention is preferably in the range from 1 to 10%, more preferably 3 to 8%, particularly 4 to 7%, and especially 5 to 6% by weight based on the total weight of the composition. The concentration of glycerine in the soap composition is preferably in the range from 1 to 15%, more preferably 3 to 12%, particularly 5 to 10%, and especially 6 to 8% by weight based on the total weight of the composition. The specific combination of sugar alcohol, particularly sorbitol, and glycerine polyol mixture reduces the formation of large opaque white crystals during a normal soap neutralisation process, enabling a transparent or translucent product to be processed on a standard soap finishing line.

The potassium salt of a carboxylic acid and/or hydroxy carboxylic acid having 6 or less carbon atoms, preferably comprises in the range from 1 to 5, more preferably 2 to 4, and especially 3 carbon atoms. The potassium salt is preferably the salt of a hydroxy carboxylic acid, and preferably comprises 1 or 2, and more preferably 1 carboxyl group(s). The potassium salt also preferably comprises 1 or 2, and more preferably 1 hydroxyl group(s). Suitable materials include the potassium salts of formic acid, lactic acid, acetic acid, citric acid, tartaric acid, malic acid, and alpha hydroxybutyric acid. In a particularly preferred embodiment of the invention, potassium lactate is employed.

The concentration of the potassium salt of a carboxylic acid and/or hydroxy carboxylic acid having 6 or less carbon atoms, preferably potassium lactate, in the soap composition according to the present invention is preferably in the range from 0.3 to 8%, more preferably 0.6 to 6%, particularly 1 to 4%, and especially 1.5 to 2% by weight based on the total weight of the composition. The presence of the aforementioned potassium salt can improve one or more of the translucency, hardness, lathering, mushing, feel and moisturizing properties of the soap composition and finished soap bar or tablet.

The concentration of water in the soap composition according to the present invention is preferably in the range from 10 to 22%, more preferably 12 to 20%, particularly 14 to 19%, and especially 16 to 18% by weight based on the total weight of the composition.

The soap composition according to the present invention may optionally contain free fatty acids, in addition to the neutralized fatty acids of the actual soap component. Preferred free fatty acids are the same types of fatty acids, as defined above, which are used to form the soap component, and therefore generally contain from 8 to 20 carbon atoms. The soap composition preferably comprises in the range from 0.5 to 10%, more preferably 1 to 5%, particularly 1.5 to 3%, and especially 2 to 2.5% by weight based on the total weight of the composition, of free fatty acids. The presence of the free fatty acids can improve both the mildness and refatting properties of the soap composition on the skin.

The soap composition suitably comprises less than 0.5%, preferably less than 0.25%, more preferably less than 0.2%, particularly less than 0.15%, and especially less than 0.1% by weight based on the total weight of the composition, of sodium chloride. In a particularly preferred embodiment of the invention, the soap composition comprises substantially no sodium chloride. The presence of sodium chloride at such low concentrations, or the complete absence thereof, in a soap composition according to the present invention, can result in improved foaming properties, and faster translucency development in the soap finishing process.

The soap composition may also contain effective amounts of other materials or functional additives. Suitable functional additives include perfumes, antioxidants, such as tocopherols BHA, BHT and the like; chelating agents, such as EDTA and the like; emulsifiers such as polyglycerol esters, eg polyglycerol monostearate; colouring agents; deodorants; dyes; emollients and skin conditioners, such as dimerized fatty acids, lanolin, cold cream, mineral oil, sorbitan esters, isopropyl myristate; enzymes; foam stabilizers; germicides; lathering agents; moisturizers; optical brighteners; dyes; pearlescers; sequestering agents; stabilizers; superfatting agents; UV absorbers; and mixtures of any two or more of these materials.

The functional additives may be used in any desired quantity to effect the desired functional characteristics, and usually minor amounts in the range from 0.01 to 5% by weight based on the total weight of the composition are used. For example, if present, (i) anti-bacterial agents and sanitizers generally comprise in the range from 0.5 to 4% by weight, (ii) emollients and skin conditioning agents generally comprise in the range from 0.5 to about 5% by weight, and (iii) perfumes, dyes and coloring agents comprise in the range from 0.2 to about 5% by weight, all based on the total weight of the composition.

In a preferred embodiment, the soap composition according to the present invention is translucent, preferably having a translucency value, measured as described herein, of greater than 15%, more preferably in the range from 20 to 80%, particularly 30 to 70%, and especially 40 to 60%.

In addition, the soap composition preferably has a total mush value, measured as described herein, of less than 30, more preferably in the range from 5 to 25, particularly 10 to 20, and especially 15 to 20 g/50 cm².

The soap composition preferably has a lather volume, measured as described herein, of greater than 30, more preferably in the range from 40 to 150, particularly 50 to 120, and especially 60 to 100 ml.

The soap composition according to the present invention may be converted into flakes, noodles, pellets, or any other suitable form or shape by methods known in the art. The converted soap composition, preferably in the form of noodles, can be mixed with other components, such as perfumes, colorants and other functional additives in an amalgamator for at least 5 minutes. The resultant mixture may be plodded or extruded into an endless bar that, after cutting into billets, can be stamped into a final soap tablet.

The invention is illustrated by the following non-limiting examples.

The following test procedures were employed;

(i) Translucency

Translucency was evaluated by measuring the light transmission of a slice of soap having a thickness of 20 mm using a reflectometer according to Dr B Lange, Type LMG 008. The result is expressed as a percentage of the light transmission of a matted glass standard. The transmission of the glass standard compared to air was 8.3% and the transmission of this standard was taken as 100%.
Translucency:
   >40%=excellent
   20 to 40%=good
   <20%=moderate
      (ii) Mush

Mush was determined by immersing a well defined portion of a soap tablet (approximate weight=45 g, and approximate surface area=70 cm²) in demineralised water at 20° C. for 2 hours. Before immersion, the weight of the soap block was measured (=W₁). After removal from the water, excess water was allowed to drip from the soap block for 1 minute, and the weight of the soap block was measured again (=W₂). All of the mush was scraped off the soap block with a plastic spatula and the soap block again reweighed (=W₃). The amount of mush is expressed in 3 different parameters, which were calculated according to the following equations (based on an immersed surface area of 50 cm²);
Total mush=(W₂−W₃)×50/immersed area
Water uptake=(W₁−W₂)×50/immersed area
Mushed soap=(W₁−W₃)×50/immersed area

Total mush is a measure of the resistance against slime formation when the soap bar is in contact with water. Water uptake and mushed soap quantity is an indication of measure of the structure of the mush.
Total mush:
   <15 g/50 cm²=excellent
   15-25 g/50 cm²=good
   >25 g/50 cm²=moderate
      (iii) Lather

Lather volume was measured by using a handwash method which closely approximates normal consumer habits. The test was carried out using a pair of surgeon's disposable latex gloves which were rinsed to remove talc. The soap tablets (approximate weight of tablets=85 g, dimensions 8 cm×5.5 cm×2.5 cm (cushion model)) to be tested were washed for 10 minutes before the test by twisting the tablet 20 times through 180° under running water at approximately 14° C. The soap tablet was then immersed in water at a temperature of 20° C., twisted 15 times through 180°, and placed back in the soap dish. Lather was than generated by rubbing the tips of the fingers of one hand against the palm of the other hand 10 times. As much lather as possible was removed from the hands by alternately gripping one hand with the other and forcing lather towards the fingertips. Accumulated lather was dislodged into a 150 ml beaker calibrated at 10 ml intervals. The whole procedure was repeated twice and the total volume of lather recorded as lather volume. Before measuring the lather volume, the lather was gently stirred to release large air pockets. The test was done in triplicate using 3 different soap tablets made from the same composition. Lather volume was calculated as an average value of the three results.
Lather volume:
   >100 ml=excellent
   50-100 ml=good
   <50 ml=moderate
      (iv) Rate of Wear

The soap tablets used in the lather volume test were weighed both before and after the test. Weighing after the test was done after the soap had dried at ambient temperature (approximately 23° C.) for at least for 24 hours. The weight difference was recorded as rate of wear and expressed in grams.
Rate of Wear:
   <3 g=excellent
   3-5 g=good
   >5 g=moderate
      (v) Hardness

A “cheese wire” with an attached weight was cut into the corner of a soap tablet, until an equilibrium position was reached. The area over which the force acts increases as the depth of the cut increases, and therefore the stress being exerted decreases until it is exactly balanced by the resistance of the soap and the wire stops moving. The stress at that point is equal to the yield stress of the soap. The time taken to reach this point was 60 seconds. After this time the weight was removed and the length of the cut measured. The yield stress was calculated from the semi-empirical formula:
Yield stress=⅜(W×98.1)/L×D N/m²
where L and D are the length of the cut and diameter of the wire in cm. W is the weight applied on the wire to obtain the cut and is given in grams.
Hardness:
   >7×10⁵ N/m²=hard
   >4<7×10⁵ N/m²=moderate
   <4×10⁵ N/m²=soft

Hardness is strongly dependent upon temperature and moisture content, and therefore measurements need to be performed under strictly controlled conditions of temperature and moisture.

### EXAMPLE 1

A soap formulation was prepared containing 53.5% by weight of sodium soap of palm oil fatty acids, 13.5% by weight of sodium soap of palm kernel oil fatty acids, 2% by weight of free fatty acids, 7.5% by weight of glycerin, 5% by weight of sorbitol, 1.5% by weight of potassium lactate, and 17% by weight of water.

The soap composition was passed 6 times through a laboratory Mazzoni M-100 duplex refiner/plodder with refining sieves of 0.5 mm and provided with a rectangular extrusion die of 45 mm×19 mm at the end of the conical outlet. The cylinder temperature was set at 30° C. and the cone temperature was 57° C. The speed of the plodder screw was fixed at 13 rpm. Soap tablets were made from the soap composition produced after each passage through the plodder. The translucency of each cycle soap tablet was measured from the corresponding billets and the results are given in Table 1.

The soap tablet made from the soap composition produced after 6 passages through the plodder, was subjected to the test procedures described herein and the results are given in Table 2.

### EXAMPLE 2

This is a comparative example not according to the present invention. The procedure of Example 1 was repeated except that the soap formulation contained no potassium lactate. The results are given in Tables 1 and 2.

TABLE 1

Translucency of Soap Tablet (%)

| Cycle | Example 1 | Example 2 (Comp.) |
|---|---|---|
| 1 | 4.4 | 4.0 |
| 2 | 6.6 | 4.4 |
| 3 | 12.9 | 6.9 |
| 4 | 27.2 | 13.4 |
| 5 | 42.2 | 29.5 |
| 6 | 54.1 | 52.1 |

TABLE 2

| Properties | Example 1 | Example 2 (Comp.) |
|---|---|---|
| (i) Translucency (%) | 54.1 | 52.1 |
| (ii) Mush | | |
| Total mush (g/50 cm²) | 21.9 | 29.2 |
| Water uptake (g/50 cm²) | 18.8 | 24.7 |
| Mushed soap (g/50 cm²) | 3.1 | 4.6 |
| (iii) Lather (ml) | 58 | 48 |
| (iv) Rate of wear (g) | 5.3 | 7.7 |
| (v) Hardness (×10⁵ N/m²) | 6.6 | 4.7 |

### EXAMPLE 3

The procedure of Example 1 was repeated except that 1.5% by weight based on the total weight of the composition of perfume oil (Green Apple (ex Fragrance Oils Ltd)) was added to the soap composition. The results are given in Tables 3 and 4.

### EXAMPLE 4

This is a comparative example not according to the present invention. The procedure of Example 2 was repeated except that 1.5% by weight based on the total weight of the composition of perfume oil (Green Apple (ex Fragrance Oils)) was added to the soap composition. The results are given in Tables 3 and 4.

TABLE 3

Translucency of Soap Tablet (%)

| Cycle | Example 3 | Example 4 (Comp.) |
|---|---|---|
| 1 | 6.6 | 2.0 |
| 2 | 8.3 | 4.5 |
| 3 | 17.8 | 9.0 |
| 4 | 35.2 | 17.0 |
| 5 | 41.4 | 34.5 |
| 6 | 50.9 | 43.7 |

TABLE 4

| Properties | Example 3 | Example 4 (Comp.) |
|---|---|---|
| (i) Translucency (%) | 50.9 | 43.7 |
| (ii) Mush | | |
| Total mush (g/50 cm²) | 17.1 | 20.0 |
| Water uptake (g/50 cm²) | 12.2 | 14.9 |
| Mushed soap (g/50 cm²) | 4.9 | 5.1 |
| (iii) Lather (ml) | 87 | 40 |
| (iv) Rate of wear (g) | 5.3 | 4.6 |
| (v) Hardness (×10⁵ N/m²) | 5.7 | 4.4 |

The above examples illustrate the improved properties of a soap composition according to the present invention.

1. A soap composition comprising (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water.

2. A soap composition according to claim 1 wherein the potassium salt is present at a concentration of 1 to 4% by weight.

3. A soap composition according to claim 1 wherein the potassium salt is potassium lactate.

4. A soap composition according to claim 1 wherein the fatty acids of the alkali metal soap are derived from palm oil and palm kernel oil.

5. A soap composition according to claim 1 wherein the alkali metal soap comprises less than 1% by weight of potassium soap.

6. A soap composition according to claim 1 wherein the molecular weight of the polyol is less than 300.

7. A soap composition according to claim 1 wherein the polyol comprises a sugar alcohol and glycerine.

8. A soap composition according to claim 1 wherein the sodium chloride content is less than 0.15% by weight.

9. A soap composition according to claim 1 additionally comprising 0.5 to 10% by weight of free fatty acids.

10. A soap composition according to claim 1 having a translucency value of greater than 15%.

11. A soap composition according to claim 1 having a total mush value of less than 30 g/50 cm².

12. A soap composition according to claim 1 having a lather volume of greater than 30 ml.

13. A soap tablet or bar comprising (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water.

14. The use of a soap composition comprising (i) 50 to 90% by weight of alkali metal soap of C₈-C₂₄ fatty acids, (ii) 3 to 25% by weight of at least one polyol, (iii) 0.1 to 10% by weight of the potassium salt of a carboxylic acid and/or hydroxy carboxylic acid having 6 or less carbon atoms, and (iv) 5 to 25% by weight of water, to form a transparent and/or translucent soap tablet or bar.
